SEQUENCE LISTING (172 sequences)

SEQ ID NO: 1 - DNA, 30 bp, artificial sequence
  primer
gccggcatatggaggtgaaagacgttctgc

SEQ ID NO: 2 - DNA, 51 bp, artificial sequence
  primer
gcggggatccttagtggtgatggtggtgatgtcagccgagccggggtgggc

SEQ ID NO: 3 - protein, 10 aa, artificial sequence
  C-terminal residues
ITTITTKDPK

SEQ ID NO: 4 - protein, 46 aa, Mouse
MAKNTAIGIDLGTTYSCVGVFQHGKVEIIANDQGNRTTPSYVAFTD

SEQ ID NO: 5 - protein, 46 aa, Rat
MAKKTAIGIDLGTTYSCVGVFQHGKVEIIANDQGNRTTPSYVAFTD

SEQ ID NO: 6 - protein, 46 aa, bovine
MAKNMAIGIDLGTTYSCVGVFQHGKVEIIANDQGNRTTPSYVAFTD

SEQ ID NO: 7 - protein, 46 aa, human
MAKRAAIGIDLGTTYSCVGVFQHGKVEIIANDQGNRTTPSYVAFTD

SEQ ID NO: 8 - protein, 47 aa, Xenopus
MATKGVAVGIDLGTTYSCVGVFQHGKVEIIANDQGNRTTPSYVAFTD

SEQ ID NO: 9 - protein, 49 aa, Arabidopsis
MAGKGEGPAIGIDLGTTYSCVGVWQHDRVEIIANDQGNRTTPSYVAFTD

SEQ ID NO: 10 - protein, 43 aa, Drosophila
MPAIGIDLGTTYSCVGVYQHGKVEIIANDQGNRTTPSYVAFTD

SEQ ID NO: 11 - protein, 44 aa, saccharomyces
MSRAVGIDLGTTYSCVAHFSNDRVEIIANDQGNRTTPSYVAFTD

SEQ ID NO: 12 - protein, 44 aa, tuberculosisH37Rv
MARAVGIDLGTTNSVVSVLEGGDPVWANSEGSRTTPSIVAFARN

SEQ ID NO: 13 - protein, 44 aa, leprae
MARAVGIDLGTTNSVVSVLEGGDPVWANSEGSRTTPSTVAFARN

SEQ ID NO: 14 - protein, 43 aa, Staph
MSKIIGIDLGTTNSCVTVLEGDEPKVIQNPEGSRTTPSWAFKN

SEQ ID NO: 15 - protein, 44 aa, E coli
GKIIGIDLGTTNSCVAIMDGTTPRVLENAEGDRTTPSIIAYTQD

SEQ ID NO: 16 - protein, 48 aa, Mouse
TERLIGDAAKNQVALNPQNTVFDAKRLIGRKFGDAWQSDMKHWPFQWN

SEQ ID NO: 17 - protein, 48 aa, Rat
TERLIGDAAKNQVALNPQNTVFDAKRLIGRKFGDPWQSDMKHWPFQWN

SEQ ID NO: 18 - protein, 49 aa, bovine
TERLIGDAAKNQVALNPQNTVFDAKRLIGRKFGDPWQSDMKEWPFRVIN

SEQ ID NO: 19 - protein, 49 aa, human
TERLIGDAAKNQVALNPQNTVFDAKRLIGRKFGDPWQSDMKHWPFQVIN

SEQ ID NO: 20 - protein, 48 aa, Xenopus
TERLIGDAAKNQVAMNPQNTVFDAKRLIGRKFNDPWQCDLKHWPFQWS

SEQ ID NO: 21 - protein, 49 aa, Arabidopsis
SERLIGDAAKNQVAMNPTNTVFDAKRLIGRRYSDPSVQADKSHWPFKWS

SEQ ID NO: 22 - protein, 49 aa, Drosophila
SERLIGDPAKNQVAMNPRNTVFDAKRLIGRKYDDPKIAEDMKHWPFKWS

SEQ ID NO: 23 - protein, 50 aa, saccharomyces
TERLIGDAAKNQAAINPHNTVFDAKRLIGRKFDDPEVTTDAKHFPFKVIS

SEQ ID NO: 24 - protein, 20 aa, tuberculosisH37Rv
GEVLVGQPAKNQAVTNVDRT

SEQ ID NO: 25 - protein, 20 aa, leprae
GEVLVGQPAKNQAVTNVDRT

SEQ ID NO: 26 - protein, 28 aa, Staph
GETQVGEVAKRQAITNPNTVQSIKRHMG

SEQ ID NO: 27 - protein, 50 aa, Ecoli
GETLVGQPAKRQAVTNPQNTLFAIKRLIGRRFQDEEVQRDVSIMPFKIIA

SEQ ID NO: 28 - protein, 49 aa, Mouse
DGDKPKVQVNYKGESRSFFPEEISSMVLTKMKEIAEAYLGHPVTNAVIT

SEQ ID NO: 29 - protein, 49 aa, Rat
DGDKPKVQVNYKGENRSFYPEEISSMVLTKMKEIAEAYLGHPVTNAVIT

SEQ ID NO: 30 - protein, 49 aa, bovine
DGDKPKVQVSYKGETKAFYPEEISSMVLTKMKEIAEAYLGHPVTNAVIT

SEQ ID NO: 31 - protein, 49 aa, human
DGDKPKVQVSYKGETKAFYPEEISSMVLTKMKEIAEAYLGYPVTNAVIT

SEQ ID NO: 32 - protein, 49 aa, Xenopus
DEGKPKVKVEYKGEEKSFFPEEISSMVLTKMKETAEAYLGHPVTNAVIT

SEQ ID NO: 33 - protein, 48 aa, Arabidopsis
GPGEKPMIWNHKGEEKQFSAEEISSIVLIKMREIAEAFLGSPVKNAWI

SEQ ID NO: 34 - protein, 49 aa, Drosophila
DGGKPKIGVEFKGEAKRFAPEEISSMVLVKMRETAEAYLGETVTDAVIT

SEQ ID NO: 35 - protein, 47 aa, saccharomyces
RDGKPWQVEYKGETKTFTPEEISSMVLSKMKETAENYLGTTVNDAWT

SEQ ID NO: 36 - protein, 43 aa, tuberculosisH37Rv
SDWSIEIDGKKYTAPEISARILMKLKRDAEAYLGEDITDAVIT

SEQ ID NO: 37 - protein, 43 aa, leprae
SDWSIEIDGKKYTAQEISARVLMKLKRDAEAYLGEDITDAVIT

SEQ ID NO: 38 - protein, 43 aa, Staph
TDYKVDIEGKSYTPQEISAMILQNLKNTAESYLGEKVDKAVIT

SEQ ID NO: 39 - protein, 46 aa, Ecoli
ADNGDAWVEVKGQKMAPPQISAEVLKKMKKTAEDYLGEPVTEAVIT

SEQ ID NO: 40 - protein, 48 aa, Mouse
VPAYFNDSQRQATKDAGVIAGLNVLRIINEPTAAAIAYGLDRTGKGER

SEQ ID NO: 41 - protein, 48 aa, Rat; identical to SEQ ID NO: 40

SEQ ID NO: 42 - protein, 48 aa, bovine; identical to SEQ ID NO: 40

SEQ ID NO: 43 - protein, 48 aa, human; identical to SEQ ID NO: 40

SEQ ID NO: 44 - protein, 48 aa, Xenopus
VPAYFNDSQRQATKDAGVLAGLNILRIINEPTAAAIAYGLDKGARGEQ

SEQ ID NO: 45 - protein, 50 aa, Arabidopsis
VPAYFNDSQRQGTKDAGVISGLNVMRIINEPTAAAIAYGLDKKASSVGEK

SEQ ID NO: 46 - protein, 48 aa, Drosophila
VPAYFNDSQRQATKDAGRIAGLNVLRIINEPTAAALAYGLDKNLQGER

SEQ ID NO: 47 - protein, 48 aa, saccharomyces
VPAYFNDSQRQATKDAGTIAGMNVLRIINEPTAAAIAYGLDKKGRAEH

SEQ ID NO: 48 - protein, 47 aa, tuberculosisH37Rv
TPAYFNDAQRQATKDAGQIAGLNVLRIVNEPTAAALAYGLDKGEKEQ

SEQ ID NO: 49 - protein, 47 aa, leprae
TPAYFNDAQRQATKEAGQIAGLNVLRIVNEPTAAALAYGLDKGEREQ

SEQ ID NO: 50 - protein, 47 aa, Staph
VPAYFNDAERQATKDAGKIAGLEVERIINEPTAAALAYGLDKTDKDE

SEQ ID NO: 51 - protein, 47 aa, E coli
VPAYFNDAQRQATKDAGRIAGLEVKRIINEPTAAALAYGLDKGTGNR

SEQ ID NO: 52 - protein, 46 aa, Mouse
NVLIFDLGGGTFDVSILTIDDGIFEVKATAGDTHLGGEDFDNRLVS

SEQ ID NO: 53 - protein, 46 aa, Rat
NVLIFDLGGGTFDVSILTIDDGIFEVKATAGDTDLGGEDFDNRLVS

SEQ ID NO: 54 - protein, 46 aa, bovine
NVLIFDLGGGTFDVSILTIDDGIFEVKATAGDTHLGGEDFDNRLVN

SEQ ID NO: 55 - protein, 46 aa, human; identical to SEQ ID NO: 54

SEQ ID NO: 56 - protein, 46 aa, Xenopus
NVLIFDLGGGTFDVSILTIDDGIFEVKATAGDTHLGGEDFDNRMVN

SEQ ID NO: 57 - protein, 46 aa, Arabidopsis
NVLIFDLGGGTFDVSLLTIEEGIFEVKATAGDTHLGGEDFDNRMVN

SEQ ID NO: 58 - protein, 47 aa, Drosophila
NVLIFDLGGGTFDVSILTIDEGSLFEVRATAGDTHLGGEDFDNRLVT

SEQ ID NO: 59 - protein, 46 aa, saccharomyces
NVLIFDLGGGTFDVSLLSIDEGVFEVKATAGDTHLGGEDFDNRLVN

SEQ ID NO: 60 - protein, 45 aa, tuberculosisH37Rv
RILVFDLGGGTFDVSLLEIGEGWEVRATSGDNHLGGDDWDQRVVD

SEQ ID NO: 61 - protein, 45 aa, leprae
TILVFDLGGGTFDVSLLEIGEGWEVRATSGDNHLGGDDWDDRIVN

SEQ ID NO: 62 - protein, 46 aa, Staph
KVLVFDLGGGTFDVSILELGDGVFEVLSTAGDNKLGGDDFDQVIID

SEQ ID NO: 63 - protein, 50 aa, E coli
TIAVYDLGGGTFDISIIEIDEVDGEKTFEVLATNGDTHLGGEDFDSRLIN

SEQ ID NO: 64 - protein, 50 aa, Mouse
HFVEEFKRKHKKDISQNKRAVRRLRTACERAKRTLSSSTQASLEIDSLFE

SEQ ID NO: 65 - protein, 50 aa, Rat; identical to SEQ ID NO: 64

SEQ ID NO: 66 - protein, 50 aa, bovine; identical to SEQ ID NO: 64

SEQ ID NO: 67 - protein, 50 aa, human; identical to SEQ ID NO: 64

SEQ ID NO: 68 - protein, 50 aa, Xenopus
HFVEEFKRKHKKDIGQNKRALRRLRTACDRAKRTLSSSSQASIEIDSLFE

SEQ ID NO: 69 - protein, 50 aa, Arabidopsis
HFVQEFKRKNKKDITGNPRALRRLRTACERAKRTLSSTAQTTIEIDSLFE

SEQ ID NO: 70 - protein, 50 aa, Drosophila
HLADEFKRKFRKDLRSNPRALRRLRTAAERAKRTLSSSTEATIEIDALFE

SEQ ID NO: 71 - protein, 50 aa, saccharomyces
HLATEFKRKTKKDISNNQRSLRRLRTAAERAKRALSSSSQTSIEIDSLFE

SEQ ID NO: 72 - protein, 50 aa, tuberculosisH37Rv
WLVDKFKGTSGIDLTKDKMAMQRLREAAEKAKIELSSSQSTSINLPYITV

SEQ ID NO: 73 - protein, 50 aa, leprae
WLVDKFKGTSGIDLTKDKMAMQRLREAAEKAKIELSSSQSTSVNLPYITV

SEQ ID NO: 74 - protein, 50 aa, Staph
YLVAEFKKENGVDLSQDKMALQRLKDAAEKAKKDLSGVSQTQISLPFISA

SEQ ID NO: 75 - protein, 50 aa, E coli
YLVEEFKKDQGIDLRNDPLAMQRLKEAAEKAKIELSSAQQTDVNLPYITA

SEQ ID NO: 76 - protein, 45 aa, Mouse
GIDFYTSITRARFEELCSDLFRGTLEPVEKALRDAKMDKAQIHDL

SEQ ID NO: 77 - protein, 45 aa, Rat
GIDFYTSITRARFEELCSDLFRGTLEPVEKALRDAKLDKAQIHDL

SEQ ID NO: 78 - protein, 45 aa, bovine
GIDFYTSITRARFEELCSDLFRSTLEPVEKALRDAKLDKAQIHDL

SEQ ID NO: 79 - protein, 45 aa, human; identical to SEQ ID NO: 78

SEQ ID NO: 80 - protein, 45 aa, Xenopus
GIDFYTAITRARFEELCSDLFRGTLEPVEKALRDAKLDKSQIHEI

SEQ ID NO: 81 - protein, 45 aa, Arabidopsis
GIDFYTTITRARFEELNMDLFRKCMEPVEKCLRDAKMDKSSVHDV

SEQ ID NO: 82 - protein, 45 aa, Drosophila
GHDFYTKVSRARFEELCADLFRNTLQPVEKALTDAKMDKGQIHDI

SEQ ID NO: 83 - protein, 45 aa, saccharomyces
GMDFYTSLTRARFEELCADLFRSTLEPVEKVLKDSKLDKSQIDEI

SEQ ID NO: 84 - protein, 50 aa, tuberculosisH37Rv
DADKNPLFLDEQLTRAEFQRITQDLLDRTRKPFQSVIADTGISVSEIDHV

SEQ ID NO: 85 - protein, 49 aa, leprae
DSDKNPLFLDEQLIRAEFQRITQDLLDRTRQPFQSWKDAGISVSEIDHV

SEQ ID NO: 86 - protein, 49 aa, Staph
GENGPLHLEVNLTRSKFEELSDSLIRRTMEPTRQAMKDAGLTNSDIDEV

SEQ ID NO: 87 - protein, 49 aa, E coli
DATGPKHMNIKVTRAKLESLVEDLVNRSIEPLKVALQDAGLSVSDIDDV

SEQ ID NO: 88 - protein, 50 aa, Mouse
VLVGGSTRIPKVQKLLQDFFNGRDLNKSINPDEAVAYGAAVQAAILMGDK

SEQ ID NO: 89 - protein, 50 aa, Rat; identical to SEQ ID NO: 88

SEQ ID NO: 90 - protein, 50 aa, bovine; identical to SEQ ID NO: 88

SEQ ID NO: 91 - protein, 50 aa, human; identical to SEQ ID NO: 88

SEQ ID NO: 92 - protein, 50 aa, Xenopus
VLVGGSTRIPKVQKLLQDFFNGRELNKSINPDEAVAYGAAVQAAILMGDK

SEQ ID NO: 93 - protein, 50 aa, Arabidopsis
VVVGGSTRIPKVQQLVQDFFNGKELCKSINPDEAVAYGAAVQAAILSGEG

SEQ ID NO: 94 - protein, 50 aa, Drosophila
VLVGGSTRIPKVEALLQEYFHGKSLNLSINPDEAVAYGAAVQAAILSGDQ

SEQ ID NO: 95 - protein, 50 aa, saccharomyces
VLVGGSTRIPKIQKLVSDFFNGKEPNRSINPDEAVAYGAAVQAAILTGDQ

SEQ ID NO: 96 - protein, 48 aa, tuberculosisH37Rv
VLVGGSTRMPAVTDLVKELTGGKEPNKGVNPDEWAVGAALQAGVLKGE

SEQ ID NO: 97 - protein, 48 aa, leprae; identical to SEQ ID NO: 96

SEQ ID NO: 98 - protein, 47 aa, Staph
ILVGGSTRIPAVQEAVKKEIGKEPNKGVNPDEWAMGAAIQGGVITGD

SEQ ID NO: 99 - protein, 48 aa, E coli
ILVGGQTRMPMVQKKVAEFFGKEPRKDVNPDEAVAIGAAVQGGVLTGD

SEQ ID NO: 100 - protein, 49 aa, Mouse
SENVQDLLLLDVAPLSLGLETAGGVMTALIKRNSTIPTKQTQTFTTYSD

SEQ ID NO: 101 - protein, 49 aa, Rat; identical to SEQ ID NO: 100

SEQ ID NO: 102 - protein, 49 aa, bovine
SENVQDLLLLDVAPLSLGLETAGGVMTALIKRNSTIPTKQTQIFTTYSD

SEQ ID NO: 103 - protein, 49 aa, human; identical to SEQ ID NO: 102

SEQ ID NO: 104 - protein, 49 aa, Xenopus
SENVQDLLLLDVAPLSLGLETAGGVMTVLIKRNTTIPTKQTQSFTTYSD

SEQ ID NO: 105 - protein, 49 aa, Arabidopsis
NEKVQDLLLLDVTPLSLGLETAGGVMTVLIPRNTTIPTKKEQIFSTYSD

SEQ ID NO: 106 - protein, 49 aa, Drosophila
TGKIQDVLLVDVAPLSLGIETAGRVMTKLIERNCRIPCKQTKTFSTYSD

SEQ ID NO: 107 - protein, 49 aa, saccharomyces
STKTQDLLLLDVAPLSLGIETAGGIMTKLIPRNSTIPTKKSETFSTYAD

SEQ ID NO: 108 - protein, 46 aa, tuberculosisH37Rv
VKDVLLLDVTPLSLGIETKGGVMTRLIERNTTIPTKRSETFTTADD

SEQ ID NO: 109 - protein, 47 aa, leprae
VKDVLLLDVTPPLSLGIETKGGVMTKLIERNTTIPTKRSETFTTADD

SEQ ID NO: 110 - protein, 46 aa, Staph
VKDVVLLDVTPLSLGIEILGGRMNTLIERNTTIPTSKSQIYSTAVD

SEQ ID NO: 111 - protein, 46 aa, E coli
VKDVLLLDVTPLSLGIETMGGVMTTLIAKNTTIPTKHSQVFSTAED

SEQ ID NO: 112 - protein, 50 aa, Mouse
NQPGVLIQVYEGERAMTRDNNLLGRFELSGIPPAPRGVPQIEVTFDIDAN

SEQ ID NO: 113 - protein, 50 aa, Rat; identical to SEQ ID NO: 112

SEQ ID NO: 114 - protein, 50 aa, bovine; identical to SEQ ID NO: 112

SEQ ID NO: 115 - protein, 50 aa, human
NQPGVLIQVYEGERAMTKDNNLLGRFELSGIPPAPRGVPQIEVTFDIDAN

SEQ ID NO: 116 - protein, 50 aa, Xenopus
NQPGVLIQVFEGERAMTKDNNLLGKFELSGIPPAPRGVPQIEVTFDIDAN

SEQ ID NO: 117 - protein, 50 aa, Arabidopsis
NQPGVLIQVYEGERARTKDNNLLGKFELSGIPPAPRGVPQITVCFDIDAN

SEQ ID NO: 118 - protein, 50 aa, Drosophila
NQPGVSIQVYEGERAMTKDNNALGTFDLSGIPPAPRGVPQIEVTFDMDAN

SEQ ID NO: 119 - protein, 50 aa, saccharomyces
NQPGVLIQVFEGERTRTKDNNLLGKFELSGIPPAPRGVPQIDVTFDIDAN

SEQ ID NO: 120 - protein, 50 aa, tuberculosisH37Rv
NQPSVQIQVYQGEREIAAHNKLLGSFELTGIPPAPRGIPQIEVTFDIDAN

SEQ ID NO: 121 - protein, 50 aa, leprae
NQPSVQIQVYQGEREIASHNKLLGSFELTGIPPAPRGVPQIEVTFDIDAN

SEQ ID NO: 122 - protein, 50 aa, Staph
NQPSVDVHVLQGERPMAADNKTLGRFQLTDIPPAERGKPQIEVTFDIDKN

SEQ ID NO: 123 - protein, 50 aa, E coli
NQSAVTIHVLQGERKRAADNKSLGQFNLDGINPAPRGMPQIEVTFDIDAD

SEQ ID NO: 124 - protein, 50 aa, Mouse
GILNVTATDKSTGKANKITITNDKGRLSKEEIERMVQEAERYKAEDEVQR

SEQ ID NO: 125 - protein, 50 aa, Rat; identical to SEQ ID NO: 124

SEQ ID NO: 126 - protein, 50 aa, bovine
GILNVTATDKSTGKANKITITNDKGRLSKEEIERMVQEAEKYKAEDEVQR

SEQ ID NO: 127 - protein, 50 aa, human
GILNVTATDKSTGKASKITITNDKGRLSKEEIERMVQEAEKYKAEDEVQR

SEQ ID NO: 128 - protein, 50 aa, Xenopus
GILNVSAVEKSSGKQNKITITNDKGRLSKEDIEKMVQEAEKYKADDDAQR

SEQ ID NO: 129 - protein, 50 aa, Arabidopsis
GILNVSAEDKTTGQKNKITITNDKGRLSKEEIEKMVQEAEKYKAEDEEHK

SEQ ID NO: 130 - protein, 50 aa, Drosophila
GILNVSAKEMSTGKAKNITIKNDKGRLSQAEIDRMVNEAEKYADEDEKHR

SEQ ID NO: 131 - protein, 50 aa, saccharomyces
GILNVSALEKGTGKSNKITITNDKGRLSKDDIDRMVSEAEKYRADDEREA

SEQ ID NO: 132 - protein, 49 aa, tuberculosisH37Rv
GIVHVTAKDKGTGKENTIRIQEGSGLSKEDIDRMIKDAEAHAEEDRKRR

SEQ ID NO: 133 - protein, 49 aa, leprae
GIVHVTAKDKGTGKENTIKIQEGSGLSKEEIDRMVKDAEAHAEEDRKRR

SEQ ID NO: 134 - protein, 49 aa, Staph
GIVNVTAKDLGTNKEQRITIQSSSSLSDEEIDRMVKDAEVNAEADKKRR

SEQ ID NO: 135 - protein, 49 aa, E coli
GILHVSAKDKNSGKEQKITIKASSGLNEDEIQKMVRDAEANAEADRKFE

SEQ ID NO: 136 - protein, 47 aa, Mouse
DRVAAKNALESYAFNMKSAVEDEGLKGKLSEADKKKVLDKCQEVISW

SEQ ID NO: 137 - protein, 47 aa, Rat
ERVAAKNALESYAFNMKSAVEDEGLKGKISEADKKKVLDKCQEVISW

SEQ ID NO: 138 - protein, 47 aa, bovine
ERVSAKNALESYAFNMKSAVEDEGLKGKISEADKKKVLDKCQEVISW

SEQ ID NO: 139 - protein, 47 aa, human; identical to SEQ ID NO: 138

SEQ ID NO: 140 - protein, 47 aa, Xenopus
ERVDAKNALESYAFNLKSMVEDENVKGKISDEDKRTISEKCTQVISW

SEQ ID NO: 141 - protein, 47 aa, Arabidopsis
KKVDAKNALENYAYNMRNTIKDEKIASKLDAADKKKIEDAIDQAIEW

SEQ ID NO: 142 - protein, 46 aa, Drosophila
QRIASRNALESYVFNVKQAVEQAGAGKLDEADKNSVLEKCNETISW

SEQ ID NO: 143 - protein, 47 aa, saccharomyces
ERVQAKNQLESYAFTLKNTINEASFKEKVGEDDAKRLETASQETIDW

SEQ ID NO: 144 - protein, 50 aa, tuberculosisH37Rv
EEADVRNQAETLVYQTEKFVKEQREAEGGSKVPEDTLNKVDAAVAEAKAA

SEQ ID NO: 145 - protein, 50 aa, leprae
EEADVRNQAETLVYQTEKFVKEQRETENGSRVPEDTLNKVEAAVAEAKTA

SEQ ID NO: 146 - protein, 45 aa, Staph
EEVDLRNEADSLVFQVEKTLTDLGENIGEEDKKSAEEKKDALKTA

SEQ ID NO: 147 - protein, 45 aa, E coli
ELVQTRNQGDHLLHSTRKQVEEAGDKLPADDKTAIESALTALETA

SEQ ID NO: 148 - protein, 42 aa, Mouse
LDSNTLADKEEFVHKREELERVCSPIISGLYQGAGAPGAGGF

SEQ ID NO: 149 - protein, 42 aa, Rat
LDSNTLAEKEEFVHKREELERVCNPIISGLYQGAGAPGAGGF

SEQ ID NO: 150 - protein, 42 aa, bovine
LDANTLAEKDEFEHKRKELEQVCNPIISRLYQGAGGPGAGGF

SEQ ID NO: 151 - protein, 42 aa, human
LDANTLAEKDEFEHKRKELEQVCNPIISGLYQGAGGPGPGGF

SEQ ID NO: 152 - protein, 46 aa, Xenopus
LENNQLAEKEEYAFQQKDLEKVCQPIITKLYQGGVPGGVPGGMPGS

SEQ ID NO: 153 - protein, 47 aa, Arabidopsis
LDGNQLAEADEFEDKMKELESLCNPIIARMYQGAGPDMGGAGGMDDD

SEQ ID NO: 154 - protein, 45 aa, Drosophila
LDSNTTAEKEEFDHRLEELTRHCSPIMTKMHQQGAGAQAGGGPGA

SEQ ID NO: 155 - protein, 50 aa, saccharomyces
LDASQAASTDEYKDRQKELEGIANPIMTKFYGAGAGAGPGAGESGGFPGS

SEQ ID NO: 156 - protein, 36 aa, tuberculosisH37Rv
LGGSDISAIKSAMEKLGQESQALGQAIYEAAQAASQ

SEQ ID NO: 157 - protein, 36 aa, leprae
LGGTDISAIKSAMEKLGQDSQALGQAIYEATQAASK

SEQ ID NO: 158 - protein, 34 aa, Staph
LEGQDIEDIKSKKEELEKVIQELSAKVYEQAAQQ

SEQ ID NO: 159 - protein, 35 aa, E coli
LKGEDKAAIEAKMQELAQVSQKLMEIAQQQHAQQQ

SEQ ID NO: 160 - protein, 19 aa, Mouse
GAQAPKGASGSGPTIEEVD

SEQ ID NO: 161 - protein, 19 aa, Rat
GAQAPKGGSGSGPTIEEVD

SEQ ID NO: 162 - protein, 19 aa, bovine
GAQGPKGGSGSGPTIEEVD

SEQ ID NO: 163 - protein, 19 aa, human
GAQGPKGGSGSGPTIEEVD

SEQ ID NO: 164 - protein, 20 aa, Xenopus
SCGAQARQGGNSGPTIEEVD

SEQ ID NO: 165 - protein, 17 aa, Arabidopsis
TPAGGSGGAGPKIEEVD

SEQ ID NO: 166 - protein, 21 aa, Drosophila
NCGQQAGGFGGYSGPTVEEVD

SEQ ID NO: 167 - protein, 21 aa, saccharomyces
MPNSGATGGGEDTGPTVEEVD

SEQ ID NO: 168 - protein, 34 aa, tuberculosisH37Rv
ATGAAHPGGEPGGAHPGSADDWDAEVVDDGREAK

SEQ ID NO: 169 - protein, 30 aa, leprae
VGGEASAPGGSNSTDDVLTRRWSTTNGSPK

SEQ ID NO: 170 - protein, 31 aa, Staph
QQQAQGANAGQNNDSTVEDAEFKEVKDDDIS

SEQ ID NO: 171 - protein, 27 aa, E coli
TAGADASANNAKDDDWDAEFEEVKDKK

SEQ ID NO: 172 - protein, 265 aa, mycobacterium tuberculosis HSP70
KDVLLLDVTPLSLGIETKGGVMTRLIERNTTIPTKRSETFTTADDNQPSVQIQVYQGEREIAAHNKLLGSFELTGIPPAPRGIPQIEVTFDIDANGIVHVTAKDKGTGKENTIRIQEGSGLSKEDIDRMIKDAEAHAEEDRKRREEADVRNQAETLVYQTEKFVKEQREAEGGSKVPEDTLNKVDAAVAEAKAALGGSDISAIKSAMEKLGQESQALGQAIYEAAQAASQATGAAHPGGEPGGAHPGSADDVVDAEVVDDGREAK

(19) United States
(12) Patent Application Publication
(10) Pub. No.: US 2006/0264609 A1
(43) Pub. Date: Nov. 23, 2006

(54) Use of heat shock proteins

(76) Inventors: Thomas Lehner, London (GB); Charles George Kelly, London (GB); Yufei Wang, London (GB); Mahavir Singh, Braunschwig (DE)

Correspondence Address: KOHN & ASSOCIATES PLLC, 30500 NORTHWESTERN HWY, STE 410, FARMINGTON HILLS, MI, 48334, US

(21) Appl. No.: 10/491,679

(22) PCT Filed: Oct. 3, 2002
(86) PCT No.: PCT/GB02/04475

§ 371 Date: Sep. 29, 2004

(30) Foreign Application Priority Data
Oct. 3, 2001 (GB) 0123756.9

(51) Int. Cl. C07K 14/47; C07H 21/04; C12P 21/06; A61K 39/00

(52) U.S. Cl. 530/350; 424/185.1; 435/69.1; 435/320.1; 435/325; 536/23.5

(57) ABSTRACT

The present invention relates to a fragment of heat shock protein that can increase the level of one or more cytokines and/or one or more CC chemokines and/or NO produced by a cell, above that caused by the corresponding full length heat shock protein. The invention also relates to the use of that fragment in the treatment or prophylaxis of a disease.

The present invention relates to the use of a heat shock protein fragment to enhance the production of cytokines and/or CC chemokines and/or nitric oxide (NO) by a cell. It also relates to the use of a heat shock protein fragment as a vaccine adjuvant, especially in the formulation of preventative or therapeutic vaccines against HIV and other microbial infection.

Heat shock proteins (HSPs) are highly conserved and widely distributed in micro-organisms as well as mammalian cells They have a number of important biological properties, especially as intracellular chaperones of proteins, and prevent proteins from aggregating when cells are stressed. HSPs have been used as carrier molecules and adjuvants, when linked to synthetic peptides.

HSP70 and HSP96 have been non-covalently bound with tumour or virus-specific peptides and been shown to have a protective effect against the specific tumour or virus (Udono et al., J. Exp. Med., 178 139-1396, 1993; Nieland et al., PNAS USA, 93 6135-6139, 1996; and Ciupitu et al., J. Exp. Med., 187 685-691, 1998). The mechanism of adjuvanticity of HSP has been elucidated by demonstrating stimulation of CC chemokines by fi11 length HSP70. The CC chemokines in turn attract T-cells, B-cells dendritic cells and macrophages.

Cytokines are proteins that mediate the induction and regulation of the immune system. They have a variety of actions, including initiation of inflammatory response, and activation of inflammatory cells. They also act on lymphocytes by stimulating growth, activation and differentiation. Cytokines are secreted by a range of cells, including activated lymphocytes and macrophages. They also have a wide Age of target cells. For example, Interleukin-12 is secreted by B cells and macrophages, and acts on activated T cells, natural killer (NK) cells and Lymphokine-activated killer (LAK) cells. Cytokines may be subdivided into groups such as lymphokines and monokines.

The term “CC chemokine” refers to any protein that has chemoattractant and proinflammatory properties, i.e. it recruits cells required for an immune response. The CC chemokines are generally of relatively low molecular weight (generally less tan 10,000). CC chemokines are produced by a variety of cell types including endothelial cells, keratinocytes, fibroblasts, natural killer (NK) cells and antigen presenting cells such as macrophages and dendritic cells. CC chemokines attract phagocytic cells and lymphocytes. Preferably the CC chemokines are β-chemokines. It is further preferred that the CC chemokines are RANTES (regulated upon activation normal T cell expressed and secreted) MIP-1α (macrophage inflammatory protein 1α) and M-1β (macrophage inflammatory protein 1β). CC chemokines attract a variety of T cells and macrophages and T cell suppressor factors which can suppress HIV and/or SIV replication. The enhanced production of CC chemokines may therefore lead to the treatment or prevention of infectious diseases such as microbial infection (including viral infections) and malignant diseases.

International patent application WO 01/45738 describes the use of full length HSPs to enhance production of one or more CC chemokines by a cell. The inventors have surprisingly found that a fragment of a HSP increases production of cytokines, especially chemokines, by a cell more than the corresponding full length HSP.

According to a first aspect of the present invention the invention provides a heat shock protein (HSP) fragment that can increase the level of one or more cytokines and/or one or more CC chemokines and/or nitric oxide (NO) produced by a cell, above that caused by the corresponding full length heat shock protein (HSP).

The term “heat shock protein” as used herein refers to any protein which exhibits increased expression in a cell when the cell is subjected to a stress. Preferably the HSP is derived from a mammalian cell more preferably a human cell. It is further preferred that the HSP is HSP70, HSP65, HSP40, HSP27, BiP, GP96, HSP60, HSP90 or HSP96. Preferably, the heat shock protein is human HSF70. The HSP may be a modified HSP, wherein the HSP has been modified to provide it with advantageous characteristics such as increased resistance to degradation

The term “full length heat shock protein” refers to a protein which comprises a substantially complete amino acid sequence of a. HSP. A “full length heat shock protein” may have been altered by minor amino acid deletions, additions or substitutions. For example, the full length HSP may be altered by between 1 and 10 amino acid deletions, additions or substitutions provided the alterations do not affect the ability of the HSP to cause the production of cytokines, CC chemokines or NO by a cell.

HSPs are commercially available. For example, HSP70 can be obtained from StressGen, Inc. and Lionex Diagnostics and Therapeutics, Braunschweig, Germany; HSP65 can be obtained from StressGen, Inc.; HSP40 can be obtained from StressGen Biotechnologies, Victoria, British Colombia Genes encoding various HSPs have been cloned and sequenced. For example, the human sequence of HSP70 has Genbank accession number M24743, mouse HSP70 has Genbank accession M35021, human HSP65 has Genbank accession number P42384 and human HSP40 has Genbank accession number D49547. Based on the known sequences of the HSPs, it would be a routine matter for one skilled in the art to obtain the desired HSP. The sequences of numerous HSP70 proteins are given in Table 1.

Furthermore, the preparation and purification of HSPs has been described in Young et al, Mol. Microbial., 6, 133-145, 1992; Mehlert et al, Mol. Microbial., 3. 125-130, 1989; and Thole et al, Infect & Immune., 5, 1466-1475, 1987.

The term “heat shock protein fragment” as used herein refers to any fragment of a HSP which can increase the levels of one or more cytokines and/or one or more CC chemokines and/or NO above the level raised by the corresponding full length HSP. The HSP fragment is preferably less than 80%, more preferably less than 70%, most preferably less than 50% of te size of the corresponding fuill length HSP. It is particularly preferred that the HSP fragment is between 10 and 300 amino acids in size, more preferably between 10 and 200 amino acids in size, most preferably between 10 and 100 amino acids in size.

Preferably the HSP fragment is a fragment of a microbial (e.g. Mycobacterium tuberculosis) HSP or a mammalian (e.g. human) HSP.

Preferably, HSP fragment has at least 40%, more preferably at least 60%, most preferably at least 80% homology to amino acid residues 359-625 or 359-610 of Mycobacterium tuberculosis HSP70. More preferably the fragmient has at least 60%, more preferably at least 70%, most preferably at least 90% homology to amino acid residues 359-459 of Mycobacterium luberctlosis HSP70. It is especially preferred that the HSP fragment has at least 80%, more preferably at least 90%, most preferably at least 95/% homology to amino acid residues 396-426 of Mycobacterium tuberculosis HSP70. The sequence of M),cobacteriurn tuberculosis HSP70 is given in Table 1. Homology can be measured using the Pileup programme, which calculates the % of amino acid substitutions and hence the homology. Preferably, the level of homology is measured using the Pileup programme having a gapweight of 8 and a gaplengthweight of 2.

It is particularly preferred that the ESP fragment consists of amino acid residues 359-625, 359-610, 359-459 or 396-426 of Mycobacterium tuberculosis HSP70. It is also preferred that the HSP fragment consists of a fragment of human HSP70, wherein the fragment corresponds to amino acid residues 359-625, 359-610, 359-459 or 396-426 of Mycobacterium tuberculosis HSP70.

The alignment of the Mycobacterium tuberculosis HSP70 with human HSP70 and other HSP70s is shown in Table 1. Based on this alignment one skilled in the art could easily determine which fragments of a HSP70 correspond to the specific fragments of Mycobacterium tuberculosis HSP70 mentioned above.

The HSP fragment preferably comprises the CD40 binding site. The position of the CD40 binding site can be easily determined by those skilled in the art.

It is also preferred that the HSP fragment does not comprise the ATPase region. The position of the ATPase region is well known to those skilled in the art.

It is also preferred that the HSP fragment does not give rise to an anti-HSP immunological response when delivered to a mammal. In order to achieve this the HSP fragment should not comprise the main antigenic epitopes of the HSP.

Preferably the HSP fragment of the invention may also comprise one or more heterologous peptides. It will be apparent to one skilled in the art that the HSP of the present invention can be used in combination with a linked or non-linked peptide or other component such as an antibody. Methods for attaching heterologous peptides are well known to those skilled in the art.

The term “a heterologous peptide” refers to any peptide that in its native state does not naturally form pat of a HSP, and is not derived from a heat shock protein. A peptide is herein defined as a polymer of amino acids and does not refer to a specific length of the product; thus, peptides, oligopeptides and proteins are included within the term peptide. The term also does not refer to or exclude postdepression modifications of the protein, for example, glycosylations, acetylations and phosphorylations. Included in the definition are peptides containing one or more analogs of an amino acid (including for example, unnatural amino acids), proteins with substituted linkages, as well as other modifications known in the art both naturally occurring and synthesised. Preferably the peptide is less that 1000 amino acid residues in length, more preferably less than 100 amino acids and length and most preferably less that 50 amino acids in length.

Preferably, the heterologous peptides are immunogenic peptides.

The term “an immunogenic peptide” refers to any peptide that can give rise to an immunogenic response within an animal body such as a mammal e.g. a human. The immunological response may be the ability of the peptide to induce an antibody or cellular response, or to stimulate a series of immune reactions in an animal that are mediated by white blood cells including lymphocytes, neutophils and monocytes.

Preferred immunogenic peptides include those derived from viruses, bacteria, protozoa, and tumours. It is particularily preferred that the immunogenic peptide is from HIV or SIV. Preferably the immunogenic peptide is gp120 or p24 from HIV.

The term “cytokine” includes any cytokine, in particular lymphokines such as interleukins and monokines. Particularly preferred cytokines include IL-12 and TNF-α.

Preferably the HSP fragment of the present invention increases production of one or more CC chemokines and/or one or more cytokines and/or NO.

Preferred CC chemokines include RANTES, MIP-1α and MIP-1β.

The term “increased production” refers to the increased production of one or more cytokines, one or more CC chemokines or NO by a cell when contacted with a HSP fragment. The increased production of the one or more cytokines and/or one or more CC chemokines may be the result of increased expression of genes encoding the one or more cytokines and the one or more CC chemokines, or maybe the result of the release of cytokines or CC chemokines from the cell. It is preferred that the production of the one or more cytokines, one or more CC chemokines or NO is enhanced by at least 20%, more preferably at least 50% and most preferably at least 80% over the level produced by a cell which is contacted with the corresponding fM length HSP.

The cell may be contacted with the HSP fragment more than once It has been found that by contacting the cell with the HSP fragment more than once, it is possible to obtain higher levels of the one or more cytokines, one or more CC chemokines and NO. The present invention therefore encompasses contacting a cell with a HSP fragment once or several times in order to obtain an enhanced production of one or more cytokines and/or one or more CC chemokines and/or NO by the cell. The term “several times” means that the cell may be contacted with the HSP fragment 2 or more times, preferably 3 to 50 times, more preferably 3 to 6 times. The interval between the repeated contacts may be from 1 day to many years depending on how long the immunological memory persists. Preferably the interval between repeated contacts is 1 month.

The present invention also provides an isolated nucleic acid molecule encoding the HSP Eminent of the present invention A nucleic acid complementary to such a nucleic acid molecule is also provided The nucleic acid may be single or double stranded, DNA or RNA, naturally or non-naturally occurring. A vector comprising the isolated nucleic acid according to the invention is also provided Vectors are molecules which serve to transfer nucleic acids of interest into a cell.

Suitable vectors include, but are not limited to, bacterial or eukaryotic vectors such as plasmids or cosmids, phage vectors such as lambda phage, viral vectors such as adenoviral vectors or baculoviral vectors. Such vectors are well known in the art.

The vector preferably comprises suitable regulatory sequences to allow the nucleic acid molecule of the invention to be expressed in a suitable host cell to produce protein encoded by the nucleic acid molecule. Typically, the vector comprises a suitable promoter and terminator sequences, or other sequences such as poly A sequences, operably Linked to the nucleic acid molecule. Such regulatory sequences are well known in the art. Also provided is a host cell comprising the vector. The cell may be bacterial, yeast or eukaryotic.

The present invention further provides a pharmaceutical composition comprising the HSP fragment according to the invention or a nucleic acid encoding the HSP fragment, in combination with a pharmaceutically acceptable excipient, carrier, adjuvant or vehicle.

The present invention also provides the fragment HSP according to the invention for use in therapy.

The present invention also provides the use of a HSP fragment according to the invention in the manufacture of a medicament for the treatment or prophylaxis of a disease. The disease may be a microbial infection, in particular a viral infection a disease of the immune system, a cancer.

Further provided is a method of treatment or prophylaxis of a disease, comprising administering to a patient in need, an effective dose of a HSP fragment. Diseases which can be treated by this method are as defined above.

The present invention also provides a method of increasing production of one or more cytokines and/or one or more CC chemokines and/or NO above the level of production brought about by the corresponding full length HSP, comprising contacting a cell with a HSP fragment according to the present invention.

The invention also provides the use of a HSP fragment according to the present invention to increase the production of one or more cytokines and/or one or more CC chemokines and/or NO above the level caused by the corresponding full length HSP.

Also provided is the use of a HSP fragment according to the present invention in the preparation of a medicament to increase the production of one or more cytokines and/or one or more CC chemokines and/or NO above the level brought about by the corresponding full length HSP for the treatment of a disease. The disease is as defined above.

The invention also provides the use of a HSP fragment according to the present invention to polarise an immune response towards a Th1 response.

Also provided is a HSP fragment according to the invention in combination with a vaccine.

Vaccines are well known to those skilled in the art and include any agent that provides a protective immune response when delivered to a mammal.

The invention further provides the use of a HSP fragment according to the invention in the preparation of a medicament to polarise the immune response towards a Th1 response.

Th cells are activated during the immune response. Following activation the Th cells divide and produce a clone of effector cells, which secrete cytokines. The cytokines have a central role in the activation of B cells, Tc cells and other immune cells. The pattern of cytokines produced by the Th cells dictates the type of immune response that is produced. A Th1 response has a cytokine profile which activates mainly T cytotoxic cells and macrophages A Th2 response activates mainly B cells.

The HSP fragment will therefore act as a Th1 adjuvant and can be used with vaccines to encourage a Th1 response.

Typically prior art adjuvants are Th2 polarising adjuvants. There is a need for Th1 polarising adjuvants. A Th1 response is more suited to infection by certain microorganisms and to diseases of the immune system In particular when dealing with a viral infection a Th1 response is preferred.

The use of a HSP fragment as defined in the present invention enables the increased production of one or more cytokines or chemokines by a cell. The production of the one or more cytokines can attract a variety of T cells and macrophages, and T cell suppressor factors which can protect the cells from infectious agents such as viruses and against tumours.

The HSP fragment of the present invention also increases the level of dendritic cell maturation, especially human dendritic cells. Dendritic cell maturation is demonstrated by upregulation of cell surface molecules such as CD83, CCR7, HLADR, CD40, CD80 and CD86. Dendritic cells are very efficient at presenting antigen, and are therefore important in the immune response.

According to the present invention the HSP fragment is delivered to a cell in order to enhance the production of one or more cytokines and/or one or more CC chemokines and/or NO by the cell The cell may be present in vito or in vivo. Preferably the cell is present in vivo and the HSP fragment, which may comprise a heterologous peptide, is delivered to an individual resulting in increased production of one or more cytokines and/or one or more CC chemokines and/or NO. Increased production of one or more cytokines and/or one or more CC chemokines and/or NO results in an immune response which can prevent microbial and viral infections, and tumour development. The HSP fragment may be administered simultaneously, subsequently or separately with a vaccine.

The HSP fragment of the present invention can be delivered to an individual in combination with any pharmaceutically acceptable carrier, adjuvant or vehicle. Pharmaceutically acceptable carriers, adjuvants and vehicles that may be used include, but are not limited to, alumina, aluminum stearate, lecithin, serum proteins, such as human serum albumin, buffer substances such as phosphates, glycine sorbic acid, potassium sorbate, partial glyceride mixtures of saturated vegetable fatty acids, water, salts or electrolytes, such as protomine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene polyoxypropylene block polymers and wool fat.

The HSP fragment of the present invention may be administered orally, parentally, by inhalation spray, topically, rectally, nasally, buccally, vaginally or by an implanted reservoir. Preferably, the HSP fragment of the present invention is administered by injection. The term “parenteral” as used herein includes subcutaneous, intracutaneous, intravenous, intramuscular, intra-articular, intrasynovial, intrasternal, intrathecal, intralesional and intracranial injection or infusion techniques.

The HSP fragment may be delivered in the form of a sterile injectable preparation, for example as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parentally-acceptable diluent or solvent, for example as a solution in 1,3-butanediol. Among the acceptable vehicles and solvents that may be employed are mannitol, water, Ringer's solution and isotonic sodium chloride solution In addition, sterile, fixed oils are conventionally employed as a solvent or suspending medium. For this purpose, any bland fixed oil may be employed including synthetic mono- or di glycerides. Fatty acids such as oleic acid and its glyceride derivatives are useful in the preparation of injectables, as are naturally pharmaceutically acceptable oils such as olive oil or caster oil, especially in their polyoxyethyated versions. These oil solutions or suspensions may also contain a long chain alcohol diluent or dispersant such as Ph.

Helv or a similar alcohol.

The HSP fragment of the present invention may also be administered as a fluid or in the form of suppositories for rectal administration. The suppository can be prepared by mixing the HSP fragment or peptides of the present invention with a suitable non-irritating excipient which is solid at room temperature but liquid at the rectal temperature and therefore will melt in the rectum to release the HSPs or peptides. Such materials include but are not limited to cocoa butter, bee's wax and polyethylene glycols.

Topical administration of the HSP fragment may be desirable when the desired treatment involves areas or organs readily accessible for topical application. For application topically to the skin, the HSP fragment should be formulated with carriers for topical administration, such as, but not limited to mineral oil, liquid petroleum, white petroleum, propylene glycol, polyoxyethylene, polyoxypropylene compounds, emulsifying wax and water. Alternatively, the HSP fragment can be formulated with a suitable lotion or cream, or dissolved in a carrier. Suitable carriers include but are not limited to mineral oil, sorbitan monosterate, polysorbate 60, cetyl esters, wax, cetearyl alcohol, 2-octyldodecanol, benzyl alcohol and water. The HSP fragment can be applied topically to the lower intestinal tract by a rectal suppository formulation or as a suitable enema formulation.

The HSP fragment of the present invention may be administered by nasal aerosol or inhalation Suitable compositions for such administration can be prepared according to techniques well known to those skilled in the art of pharmaceutical formulation and can be prepared as solutions in saline, employing benzyl alcohol or other preservatives, absorbtion promoters to enhance bio-availability, fluorocarbons, arid/or other solublising other dispersing agents known in the art.

The following examples, with reference to the figures, are offered by way of illustration and are not intended to limit the invention in any manner.

The figures show:

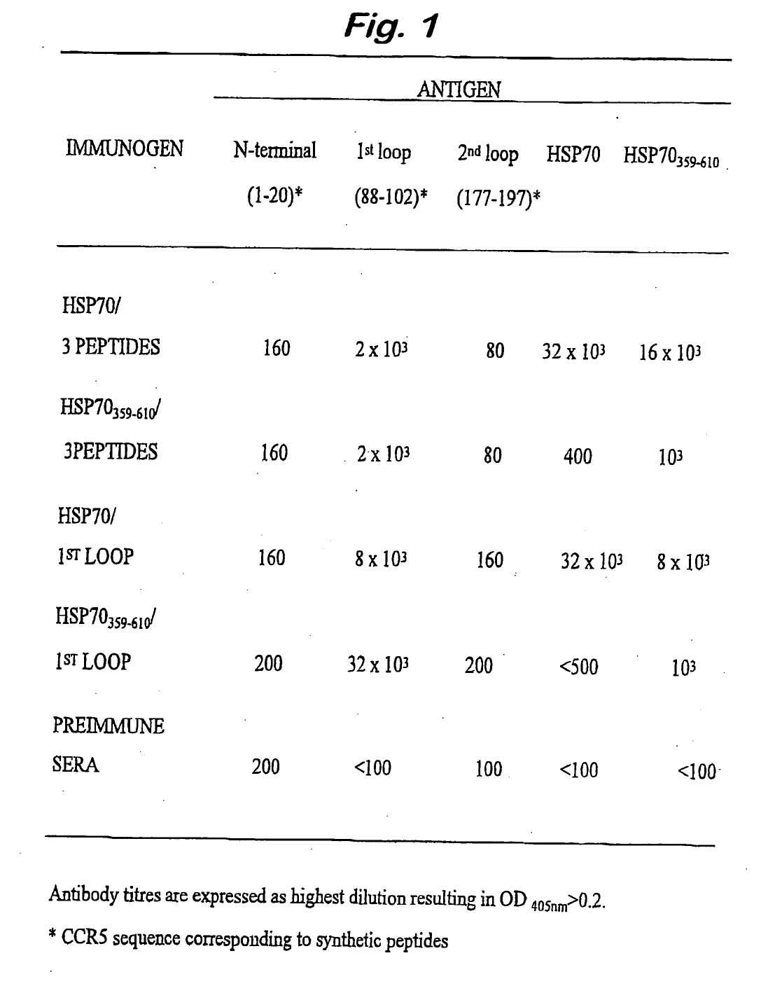

FIG. 1 shows serum antibody responses in C57BL/6J mice after immunisation with synthetic peptides non-covalently complexed with HSP70 or HSP70₃₅₉₋₆₁₀.

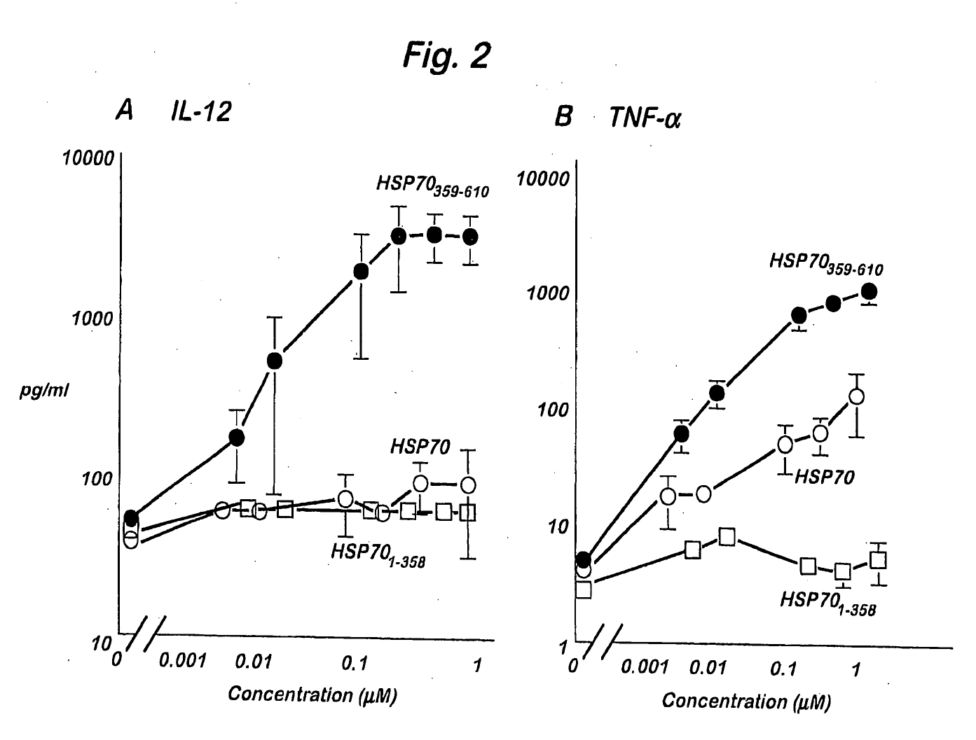

FIG. 2 shows the effects of HSP70, HSP70₁₋₃₅₈ and HSP70₃₅₉₋₆₁₀ on production of IL-12and THP-α by THP1 cells.

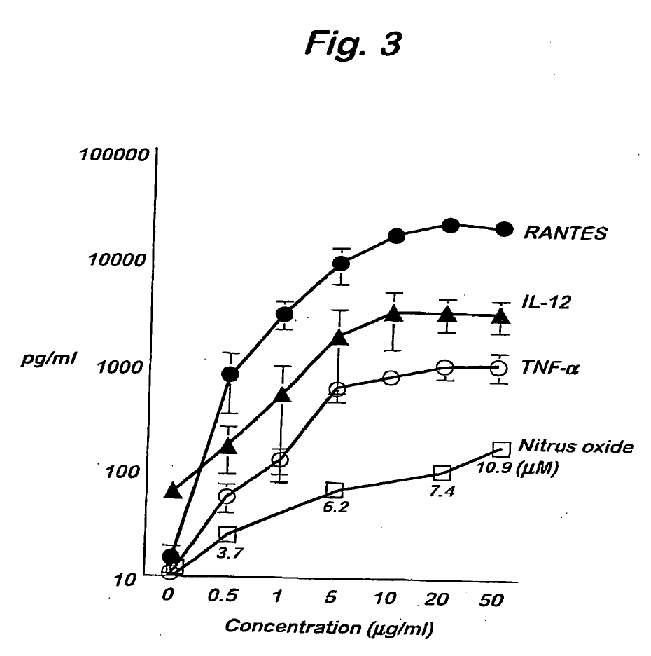

FIG. 3 shows the effects of HSP70₃₅₉₋₆₁₀ on the production of RANTES, IL-12 and TNF-α by monocytic THP1 cells.

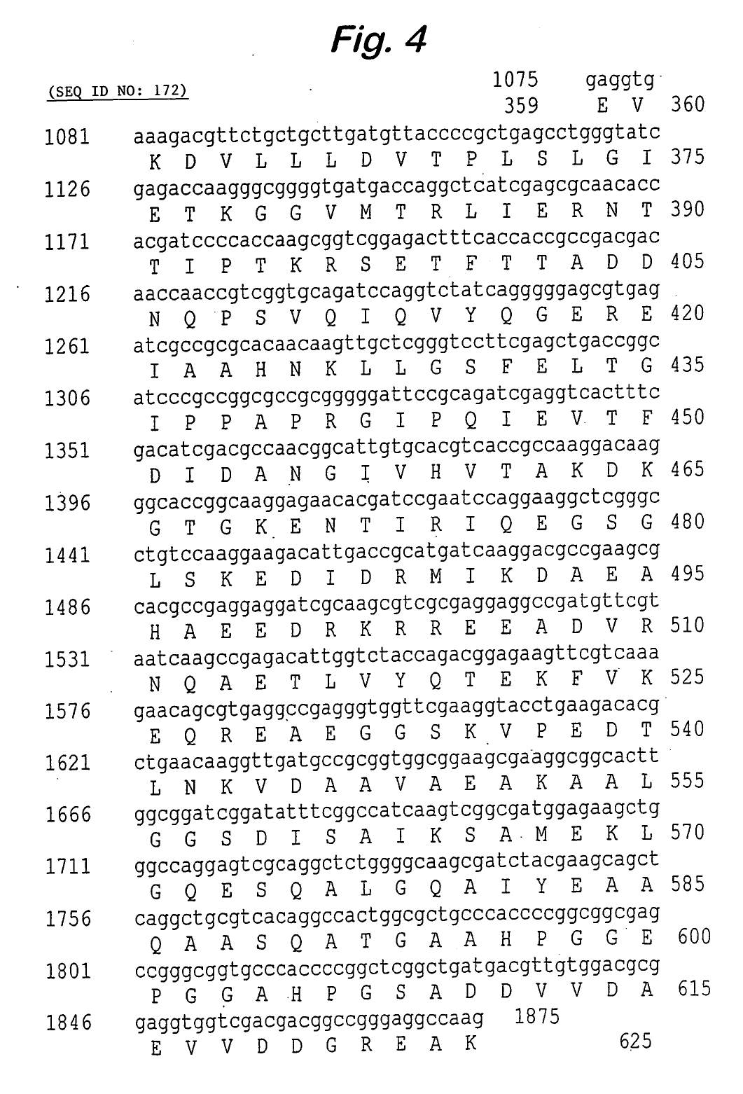

FIG. 4 shows the nucleic acid and amino acid sequences of Mycobacterium tuberculosis HSP70

### EXAMPLES

The production of the functional fragment by recombinant DNA techniques is described below.

### Example 1

### Construction of an expression plasmid and production strain for HSP70₃₅₉₋₆₁₀ from Mycobacterium tuberculosis

Amplification of DNA Fragment Encoding HSP70₃₅₉₋₆₁₀

To amplify the region of the M. tuberculosis HSP70 gene by polymerase chain reaction, the primers (20 pmol each) 5′-GCC GGC ATA TGG AGG TGA AAG ACG TTC TGC-3′ and 5′-GCG GGG ATC CTT AGT GGT GAT GGT GGT GAT GTC AGC CGA GCC GGG GTG GGC-3′ were used together with the plasmid pKAM2101 as template. This is a plasmid containing the M. tuberculosis HSP70 gene and is available from the WHO antigen bank maintained by Professor M. Singh at Gesellschaft fur Biotechnologische Forschung (GBF), Braunschweig, Germany. The reaction was performed using Taq-polymerase (Qiagen) and conditions were according to manufacturer's instructions.

Construction of Expression Vector pLEXWO27-2

The PCR product was purified using the QIA Extraction kit (Qiagen) and was digested with BamHI for 2 h. Following extraction with phenol for inactivation of the restriction endonuclease, digested DNA was recovered by ethanol precipitation Digested DNA was then further cleaved, using standard conditions, with NdeI which was subsequently inactivated by heat treatment. The same procedure was used to prepare vector pJLA603. The digested PCR product was ligated to pJLA603 (see Schauder B. et al 1 987 Gene, vol 52 p279-283 using T4-ligase (Roche) according to manufacturer's instructions.

The ligation-mixture was directly transformed into CₐCl₂ competent Escherichia coli DH5α cells and spread onto selective medium. Plasmids were reisolated from the clones and analyzed by restriction with NdeI and BamHI. Two plasmids containing the coding region of the peptide binding domain were introduced into expression strain. E. coli CAG629 by electroporation. This CAG strain is described by Singh M, et al, The Mycobacterium tuberculosis 39-kDA antigen: overproduction in Escherischia coli, purification and characterisation, Gene 117:5360, 1992. Other strains can be used as alternatives e.g. E. coli BL21.

Transformants were again analyzed by restriction of the reisolated plasmids. The expression level of HSP70₃₅₉₋₆₁₀ was analyzed, after heat induction, by SDS-PAGE.

The cloned insert of pLEXWO27-2 was confirmed by DNA sequence analysis. The sequence is shown in FIG. 1. As a result of the cloning procedures used, the construct HSP70₃₅₉₋₆₁₀ was expressed with an additional 10 residues (ITTITKDPK, not shown in FIG. 1) at the C-terminal and an additional single residue (M, also not shown in FIG. 1). These residues are not part of the sequence of M. tuberculosis HSP70 but do not affect the activity of the specified fragment.

### Example 2

### Preparation of Recombinant HSP70₃₅₉₋₆₁₀

Bacterial Culture

For production of HSP70₃₅₉₋₆₁₀, E. coli strain CAG629/pWO27-2 (i.e. E. coli strain CAG629 transformed with pLEXWO27-2) was grown in 1 L LB-medium containing 100 μg ampicillin per mL. The culture was inoculated with an OD600 of approx. 0.15 and incubated at 30° C. and 180 rpm. After reaching OD₆₀₀=0.3, protein expression was induced by shifting the temperature to 42° C. Cells were harvested after 3.5 h at OD₆₀₀=1.2. The cell pellets were stored at −20° C. or used directly for purification of HSP70₃₅₉₋₆₁₀.

Purification of HSP70₃₅₉₋₆₁₀

HisBind Quick Columns (Novagen) were used according to the manufacturer's instructions for purification of HSP70₃₅₆₋₆₁₀. Cell pellets (2 g) harvested as above, were resuspended in 10 mL binding buffer without imidazole and disrupted by sonication. The crude extract was centrifuged for 10 min at 4000×g. The supernatant was then loaded onto a HisBind Quick Column After washing the column with 30 mL binding buffer without imidazole HSP70₃₅₉₋₆₁₀ was eluted with 15 mL buffer containing 150 mM imidazole. The purified polypeptide was analysed by SDS-PAGE.

### Example 3

### Stimulation of RANTES IL-12, TNF-α Nitric Oxide

THP1 cells (2×10⁵ ml) were cultured in 24 well plates and incubated with various concentrations of HSP70, HSP70₃₅₉₋₆₁₀ or HSP70₁₋₃₅₈ (N-terminal domain). To rule out the effect of any remaining contamination with LPS in the HSP70 preparation, 50 [g/ml of polymyxin B was added to the cultures of monocytes stimulated with either HSP70 or LPS. After 3-5 days, the supernatant was used to assay RANTES, IL12, TNF-α Nitric oxide. In contrast to intact HSP70 or HSP70₁₋₃₅₈, HSP70₃₅₉₋₆₁₀ stimulated IL12 production (FIG. 2). HSP70₃₅₉₋₆₁₀ also stimulated increased production of TNF-α, RANTES and NO compared with intact HSP70 (FIGS. 2 and 3).

Properties of HSP70₃₅₉₋₆₁₀

To compare the properties of HSP70₃₅₉₋₆₁₀ with that of intact HSP70, mice were immunised with synthetic peptides corresponding to extracellular regions of the chemokine receptor CCR5 bound non-covalently to HSP70₃₅₉₋₆₁₀ or to intact HSP70. Groups of 4 C57BL/6J mice were immunised intraperitoneally with a boost after 4 weeks and the serum antibody response was determined by ELISA. Following immunisation with HSP70 non-covalently associated with a mixture of synthetic peptides corresponding to sequences of the N-terminal, 1^{st} loop and 2^{nd} loop of CCR5, serum antibody responses were induced principally to the 1^{st} loop (1 in 2,000) as well as to HSP70 (1 in 32,000) and HSP70₃₅₉₋₆₁₀ (1 in 16,000) (Table 1). Serum antibody titres to the N-terminal and loop 2 peptides were not significantly greater than those of the preimmune sera (Table 1). Similar responses were induced when mice were immunised with the peptides bound non-covalently to HSP70₃₅₉₋₆₁₀ although in this case, the response to intact HSP70 (<1 in 500) or HSP70₃₅₉₋₆₁₀ (1 in 1,000) was considerably lower. Mice were also immunised with HSP70 or HSP70₃₅₉₋₆₁₀ non-covalently associated solely with the most immunogenic 1^{st} loop peptide. As before, immunisation with peptide complexed with HSP70 induced responses to the is loop peptide (1 in 8,000), HSP70 (1 in 32,000) and HSP70₃₅₉₋₆₁₀ (1 in 8000). Immunisation with HSP70₃₅₉₋₆₁₀ resulted in an increased serum antibody response to the 1^{st} loop peptide (1 in 32,000) but considerably reduced responses to both HSP70 and HSP70₃₅₉₋₆₁₀.

In summary the HSP fragment has the following advantages.

a) It is effective both by systemic and mucosal administration.

b) It induces Th-1 polarisation of the immune response and therefore elicits CD8⁺ T-cell, CD4⁺T cell and antibody responses.

c) It has a chaperone function that may impart desirable conformation to peptides.

d) It stimulates production of CC chemokines that block and downregulate the CCR5 receptor, thereby having a specific anti-HIV effect.

e) The fragment induces maturation of dendritic cells, that facilitates antigen presentation to T cells.

All documents cited above are incorporated herein by reference.

TABLE 1

!!AA_MULTIPLE_ALIGNMENT 1.0
Pileup of: @hsp70-listfile.txt
Symbol comparison table: GenRunData:blosum62.cmp CompCheck: 1102
GapWeight: 8
GapLengthweight: 2
Hsp70-proteins.msf MSF: 686 Type: P Sep. 27, 2002 14:33 Check: 81 . . .

| | | | |
|---|---|---|---|
| Name: Mouse | Len: 686 | Check: 5051 | Weight: 1.00 |
| Name: Rat | Len: 686 | Check: 9373 | Weight: 1.00 |
| Name: bovine | Len: 6B6 | Check: 4580 | Weight: 1.00 |
| Name: human | Len: 686 | Check: 5101 | Weight: 1.00 |
| Name: Xenopus | Len: 686 | Check: 1574 | Weight: 1.00 |
| Name: Arabidopsis | Len: 686 | Check: 3665 | Weight: 1.00 |
| Name: Drosophila | Len: 686 | Check: 9083 | Weight: 1.00 |
| Name: saccharomyces | Len: 686 | Check: 9781 | Weight: 1.00 |
| Name: tuberculosisH37Rv | Len: 686 | Check: 6358 | Weight: 1.00 |
| Name: leprae | Len: 686 | Check: 1476 | Weight: 1.00 |
| Name: Staph | Len: 686 | Check: 9782 | Weight: 1.00 |
| Name: Ecoli | Len: 686 | Check: 4257 | Weight: 1.00 |
| // | | | |

| | |
|---|---|
| | 1 50 |
| Mouse | ---MAKNTAI GIDLGTTYSC VGVFQHGKVE IIANDQGNRT TPSYVAFT.D |
| Rat | ---MAKNTAI GIDLGTTYSC VGVFQHGKVE IIANDQGNRT TPSYVAFT.D |
| bovine | ---MAKNMAI GIDLGTTYSC VGVFQHGKVE IIANDQGNRT TPSYVAFT.D |
| human | ---MAKAAAI GIDLGTTYSC VGVFQHGKVE IIANDQGNRT TPSYVAFT.D |
| Xenopus | --MATKGVAV GIDLGTTYSC VGVFQHGKVE IIANDQGNRT TPSYVAFT.D |
| Arabidopsis | MAGKGEGPAI GIDLGTTYSC VGVWQHDRVE IIANDQGNRT TPSYVAFT.D |
| Drosophila | ------MPAI GIDLGTTYSC VGVYQHGKVE IIANDQGNRT TPSYVAFT.D |
| saccharomyces | -----MSRAV GIDLGTTYSC VAHFSNDRVE IIANDQGNRT TPSYVAFT.D |
| tuberculosisH37Rv | -----MARAV GIDLGTTNSV VSVLEGGDPV VVANSEGSRT TPSIVAFARN |
| leprae | -----MARAV GIDLGTTNSV VSVLEGGDPV VVANSEGSRT TPSTVAFARN |
| Staph | -----MSKII GIDLGTTNSC VTVLEGDEPK VIQNPEGSRT TPSVVAF.KN |
| Ecoli | ------GKII GIDLGTTNSC VAIMDGTTPR VLENAEGDRT TPSIIAYTQD |
| | 51 100 |
| Mouse | TERLIGDAAK NQVALNPQNT VFDAKRLIGR KFGDAVVQSD MKHWPFQVVN |
| Rat | TERLIGDAAK NQVALNPQNT VFDAKRLIGR KFGDPVVQSD MKHWPFQVVN |
| bovine | TERLIGDAAK NQVALNPQNT VFDAKRLIGR KFGDPVVQSD MKEWPFRVIN |
| human | TERLIGDAAK NQVALNPQNT VFDAKRLIGR KFGDPVVQSD MKHWPFQVIN |
| Xenopus | TERLIGDAAK NQVAMNPQNT VFDAKRLIGR KFGDPVVQCD LKHWPFKVVS |
| Arabidopsis | SERLIGDAAK NQVAMNPTNT VFDAKRLIGR RYSDPSVQAD KSHWPFKVVS |
| Drosophila | SERLIGDPAK NQVAMNPRNT VFDAKRLIGR KYDDPKIAED MKHWPFKVVS |
| saccharomyces | TERLIGDAAK NQAAINPHNT VFDAKRLIGR KFDDPEVTTD AKHFPFKVIS |
| tuberculosisH37Rv | GEVLVGQPAK NQAVTNVDRT VRSVKRHMS. .......... .......... |
| leprae | GEVLVGQPAK NQAVTNVDRT IRSVKRHMG. .......... .......... |
| Staph | GETQVGEVAK RQAITN.PNT VQSIKRHMG. .......... .......... |
| Ecoli | GETLVGQPAK RQAVTNPQNT LFAIKRLIGR RFQDEEVQRD VSIMPFKIIA |
| | 101 150 |
| Mouse | .DGDKPKVQV NYKGESRSFF PEEISSMVLT KMKEIAEAYL GHPVTNAVIT |
| Rat | .DGDKPKVQV NYKGENRSFY PEEISSMVLT KMKEIAEAYL GHPVTNAVIT |
| bovine | .DGDKPKVQV SYKGETKAFY PEEISSMVLT KMKEIAEAYL GHPVTNAVIT |
| human | .DGDKPKVQV SYKGETKAFY PEEISSMVLT KMKEIAEAYL GYPVTNAVIT |
| Xenopus | .DEGKPKVKV EYKGEEKSFF PEEISSMVLT KMKETAEAYL GHPVTNAVIT |
| Arabidopsis | GPGEKPMIVV NHKGEEKQFS AEEISSIVLI KMREIAEAFL GSPVKNAVVI |
| Drosophila | .DGGKPKIGV EFKGEAKRFA PEEISSMVLV KMRETAEAYL GETVTDAVIT |
| saccharomyces | RDG.KPVVQV EYKGETKTFT PEEISSMVLS KMKETAENYL GTTVNDAVVT |
| tuberculosisH37Rv | ...SDWSIEI ....DGKKYT APEISARILM KLKRDAEAYL GEDITDAVIT |
| leprae | ...SDWSIES ....DGKKYT AQEISARVLM KLKRDAEAYL GEDITDAVIT |
| Staph | ...TDYKVDI ....EGKSYT PQEISAMILQ NLKNTAESYL GEKVDKAVIT |
| Ecoli | ADNGDAWVEV ....KGQKMA PPQISAEVLK KMKKTAEDYL GEPVTEAVIT |
| | 151 200 |
| Mouse | VPAYFNDSQR QATKDAGVIA GLNVLRIINE PTAAAIAYGL DRTGK..GER |
| Rat | VPAYFNDSQR QATKDAGVIA GLNVLRIINE PTAAAIAYGL DRTGK..GER |
| bovine | VPAYFNDSQR QATKDAGVIA GLNVLRIINE PTAAAIAYGL DRTGK..GER |
| human | VPAYFNDSQR QATKDAGVIA GLNVLRIINE PTAAAIAYGL DRTGK..GER |
| Xenopus | VPAYFNDSQR QATKDAGVLA GLNILRIINE PTAAAIAYGL DKGAR..GEQ |
| Arabidopsis | VPAYFNDSQR QGTKDAGVIS GLNVMRIINE PTAAAIAYGL DKKASSVGEK |
| Drosophila | VPAYFNDSQR QATKDAGRIA GLNVLRIINE PTAAAIAYGL DK..NLQGER |
| saccharomyces | VPAYFNDSQR QATKDAGTIA GMNVLRIINE PTAAAIAYGL DKKGR..AEH |
| tuberculosisH37Rv | TPAYFNDAQR QATKDAGQIA GLNVLRIVNE PTAAALAYGL DKGEK...EQ |
| leprae | TPAYFNDAQR QATKEAGQIA GLNVLRIVNE PTAAALAYGL DKGER...EQ |
| Staph | VPAYFNDAER QATKDAGKIA GLEVERIINE PTAAALAYGL DKTDK...DE |
| Ecoli | VPAYFNDAQR QATKDAGRIA GLEVKRIINE PTAAALAYGL DKGTG...NR |
| | 201 250 |
| Mouse | NVLIFDLGGG TFDVSILTID DG....IFEV KATAGDTHLG GEDFDNRLVS |
| Rat | NVLIFDLGGG TFDVSILTID DG....IFEV KATAGDTDLG GEDFDNRLVS |
| bovine | NVLIFDLGGG TFDVSILTID DG....IFEV KATAGDTHLG GEDFDNRLVN |
| human | NVLIFDLGGG TFDVSILTID DG....IFEV KATAGDTHLG GEDFDNRLVN |
| Xenopus | NVLIFDLGGG TFDVSILTID DG....IFEV KATAGDTHLG GEDFDNRMVN |
| Arabidopsis | NVLIFDLGGG TFDVSLLTIE EG....IFEV KATAGDTHLG GEDFDNRMVN |
| Drosophila | NVLIFDLGGG TFDVSILTID EG...SLFEV RATAGDTHLG GEDFDNRLVT |
| saccharomyces | NVLIFDLGGG TFDVSLLSID EG....VFEV KATAGDTHLG GEDFDNRLVN |
| tuberculosisH37Rv | RILVFDLGGG TFPVSLLEI. ...GEGVVEV RATSGDNHLG GDDWDQRVVD |
| leprae | TILVFDLGGG TFDVSLLEI. ...GEGVVEV RATSGDNHLG GDDWDDRIVN |
| Staph | KVLVFDLGGG TFDVSILEL. ...GDGVFEV LSTAGDNKLG GDDFDQVIID |
| Ecoli | TIAVYDLGGG TFDISIIEID EVDGEKTFEV LATNGDTHLG GEDFDSRLIN |
| | 251 300 |
| Mouse | HFVEEFKRKH KKDISQNKRA VRRLRTACER AKRTLSSSTQ ASLEIDSLFE |
| Rat | HFVEEFKRKH KKDISQNKRA VRRLRTACER AKRTLSSSTQ ASLEIDSLFE |
| bovine | HFVEEFKRKH KKDISQNKRA VRRLRTACER AKRTLSSSTQ ASLEIDSLFE |
| human | HFVEEFKRKH KKDISQNKRA VRRLRTACER AKRTLSSSTQ ASLEIDSLFE |
| Xenopus | HFVEEFKRKH KKDIGQNKRA LRRLRTACDR AKRTLSSSSQ ASIEIDSLFE |
| Arabidopsis | HFVQEFKRKN KKDITGNPRA LRRLRTACER AKRTLSSTAQ TTIEIDSLFE |
| Drosophila | HLADEFKRKF RKDLRSNPRA LRRLRTAAER AKRTLSSSTE ATIEIDALFE |
| saccharomyces | HLATEFKRKT KKDISNNQRS LRRLRTAAER AKRALSSSSQ TSIEIDSLFE |
| tuberculosisH37Rv | WLVDKFKGTS GIDLTKDKMA MQRLREAAEK AKIELSSSQS TSINLPYITV |
| leprae | WLVDKFKGTS GIDLTKDKMA MQRLREAAEK AKIELSSSQS TSVNLPYITV |
| Staph | YLVAEFKKEN GVDLSQDKMA LQRLKDAAEK AKKDLSGVSQ TQISLPFISA |
| Ecoli | YLVEEFKKDQ GIDLRNDPLA MQRLKEAAEK AKIELSSAQQ TDVNLPYITA |
| | 301 350 |
| Mouse | GID.....FY TSITRARFEE LCSDLFRGTL EPVEKALRDA KMDKAQIHDL |
| Rat | GID.....FY TSITRARFEE LCSDLFRGTL EPVEKALRDA KLDKAQIHDL |
| bovine | GID.....FY TSITRARFEE LCSDLFRSTL EPVEKALRDA KLDKAQIHDL |
| human | GID.....FY TSITRARFEE LCSDLFRSTL EPVEKALRDA KLDKAQIHDL |
| Xenopus | GID.....FY TAITRARFEE LCSDLFRGTL EPVEKALRDA KLDKSQIHEI |
| Arabidopsis | GID.....FY TTITRARFEE LNMDLFRKCM EPVEKCLRDA KMDKSSVHDV |
| Drosophila | GHD.....FY TKVSRARFEE LCADLFRNTL QPVEKALTDA KMDKGQIHDI |
| saccharomyces | GMD.....FY TSLTRARFEE LCADLFRSTL EPVEKVLKDS KLDKSQIDEI |
| tuberculosisH37Rv | DADKNPLFLD EQLTRAEFQR ITQDLLDRTR KPFQSVIADT GISVSEIDEV |
| leprae | DSDKNPLFLD EQLIRAEFQR ITQDLLDRTR QPFQSVVKDA GISVSEIDHV |
| Staph | .GENGPLHLE VNLTRSKFEE LSDSLIRRTM EPTRQAMKDA GLTNSDIDEV |
| Ecoli | DA.TGPKHMN IKVTRAKLES LVEDLVNRSI EPLKVALQDA GLSVSDIDDV |
| | 351 400 |
| Mouse | VLVGGSTRIP KVQKLLQDFF NGRDLNKSIN PDEAVAYGAA VQAAILMGDK |
| Rat | VLVGGSTRIP KVQKLLQDFF NGRDLNKSIN PDEAVAYGAA VQAAILMGDK |
| bovine | VLVGGSTRIP KVQKLLQDFF NGRDLNKSIN PDEAVAYGAA VQAAILMGDK |
| human | VLVGGSTRIP KVQKLLQDFF NGRDLNKSIN PDEAVAYGAA VQAAILMGDK |
| Xenopus | VLVGGSTRIP KVQKLLQDFF NGRELNKSIN PDEAVAYGAA VQAAILMGDK |
| Arabidopsis | VLVGGSTRIP KVQQLLQDFF NGKELNKSIN PDEAVAYGAA VQAAILMGEG |
| Drosophila | VLVGGSTRIP KVEALLQEYF HGKSLNLSIN PDEAVAYGAA VQAAILSGDQ |
| saccharomyces | VLVGGSTRIP KIQKLVSDFF NGKEPNRSIN PDEAVAYGAA VQAAILTGDQ |
| tuberculosisH37Rv | VLVGGSTRMP AVTDLVKELT GGKEPNKGVN PDEVVAVGAA LQAGVLKGE. |
| leprae | VLVGGSTRMP AVTDLVKELT GGKEPNKGVN PDEVVAVGAA LQAGVLKGE. |
| Staph | ILVGGSTRIP AVQEAVKKEI .GKEPNKGVN PDEVVAMGAA IQGGVITGD. |
| Ecoli | ILVGGQTRMP MVQKKVAEFP .GKEPRKDVN PDEAVAIGAA VQGGVLTGD. |
| | 401 450 |
| Mouse | SENVQDLLLL DVA.PLSLGL ETAGGVMTAL IKRNSTIPTK QTQTFTTYSD |
| Rat | SENVQDLLLL DVA.PLSLGL ETAGGVMTAL IKRNSTIPTK QTQTFTTYSD |
| bovine | SENVQDLLLL DVA.PLSLGL ETAGGVMTAL IKRNSTIPTK QTQTFTTYSD |
| human | SENVQDLLLL DVA.PLSLGL ETAGGVMTAL IKRNSTIPTK QTQTFTTYSD |
| Xenopus | SENVQDLLLL DVA.PLSLGL ETAGGVMTVL IKRNTTIPTK QTQTFTTYSD |
| Arabidopsis | NEKVQDLLLL DVT.PLSLGL ETAGGVMTVL IPRNTTIPTK KEQIFSTYSD |
| Drosophila | TGKIQDVLLV DVA.PLSLGI ETAGRVMTKL IERNCRIPCK QTKTFSTYSD |
| saccharomyces | STKTQDLLLL DVA.PLSLGI ETAGGIMTKL IPRNSTIPTK KSETFSTYAD |
| tuberculosisH37Rv | ...VKDVLLL DVT.PLSLGI ETKGGVMTRL IERNTTIPTK RSETFTTADD |
| leprae | ...VKDVLLL DVTPPLSLGI ETKGGVMTKL IERNTTIPTK RSETFTTADD |
| Staph | ...VKDVVLL DVT.PLSLGI EILGGRMNTL IERNTTIPTS KSQIYSTAVD |
| Ecoli | ...VKDVLLL DVT.PLSLGI ETMGGVMTTL IAKNTTIPTK HSQVFSTAED |
| | 451 500 |
| Mouse | NQPGVLIQVY EGERAMTRDN NLLGRFELSG IPPAPRGVPQ IEVTFDIDAN |
| Rat | NQPGVLIQVY EGERAMTRDN NLLGRFELSG IPPAPRGVPQ IEVTFDIDAN |
| bovine | NQPGVLIQVY EGERAMTRDN NLLGRFELSG IPPAPRGVPQ IEVTFDIDAN |
| human | NQPGVLIQVY EGERAMTKDN NLLGRFELSG IPPAPRGVPQ IEVTFDIDAN |
| Xenopus | NQPGVLIQVF EGERAMTKDN NLLGKFELSG IPPAPRGVPQ IEVTFDIDAN |
| Arabidopsis | NQPGVLIQVY EGERARTKDN NLLGKFELSG IPPAPRGVPQ ITVCFDIDAN |
| Drosophila | NQPGVSIQVY EGERAMTKDN NALGTFDLSG IPPAPRGVPQ IEVTFDMDAN |
| saccharomyces | NQPGVLIQVF EGERTRTKDN NLLGKFELSG IPPAPRGVPQ IDVTFDIDAN |
| tuberculosisH37Rv | NQPSVQIQVY QGEREIAAHN KLLGSFELTG IPPAPRGIPQ IEVTFDIDAN |
| leprae | NQPSVQIQVY QGEREIASHN KLLGSFELTG IPPAPRGVPQ IEVTFDIDAN |
| Staph | NQPSVDVHVL QGERPMAADN KTLGRFQLTD IPPAERGKPQ IEVTFDIDKN |
| Ecoli | NQSAVTIHVL QGERKRAADN KSLGQFNLDG INPAPRGMPQ IEVTFDIDAD |
| | 501 550 |
| Mouse | GILNVTATDK STGKANKITI TNDKGRLSKE EIERMVQEAE RYKAEDEVQR |
| Rat | GILNVTATDK STGKANKITI TNDKGRLSKE EIERMVQEAE RYKAEDEVQR |
| bovine | GILNVTATDK STGKANKITI TNDKGRLSKE EIERMVQEAE KYKAEDEVQR |
| human | GILNVTATDK STGKASKITI TNDKGRLSKE EIERMVQEAE KYKAEDEVQR |
| Xenopus | GILNVSAVEK SSGKQNKITI TNDKGRLSKE DIEKMVQEAE KYKADDDAQR |
| Arabidopsis | GILNVSAEDK TTGQKNKITI TNDKGRLSKE EIEKMVQEAE KYKAEDEEHK |
| Drosophila | GILNVSAKEM STGKAKNITI KNDKGRLSQA EIDRMVNEAE KYADEDEKHR |
| saccharomyces | GILNVSALEK GTGKSNKITI TNDKGRLSKD DIDRMVSEAE KYRADDEREA |
| tuberculosisH37Rv | GIVHVTAKDK GTGKENTIRI QEGSG.LSKE DIDRMIKDAE AHAEEDRKRR |
| leprae | GIVHVTAKDK GTGKENTIKI QEGSG.LSKE EIDRMVKDAE AHAEEDRKRR |
| Staph | GIVNVTAKDL GTNKEQRITI QSSSS.LSDE EIDRMVKDAE VNAEADKKRR |
| Ecoli | GILHVSAKDK NSGKEQKITI KASSC.LNED EIQKMVRDAE ANAEADRKFE |
| | 551 600 |
| Mouse | DRVAAKNALE SYAFNMKSAV EDEGLK...G KLSEADKKKV LDKCQEVISW |
| Rat | ERVAAKNALE SYAFNMKSAV EDEGLK...G KISEADKKKV LDKCQEVISW |
| bovine | ERVSAKNALE SYAFNMKSAV EDEGLK...G KISEADKKKV LDKCQEVISW |
| human | ERVSAKNALE SYAFNMKSAV EDEGLK...G KISEADKKKV LDKCQEVISW |
| Xenopus | ERVDAKNALE SYAFNLKSMV EDENVK...G KISDEDKRTI SEKCTQVISW |
| Arabidopsis | KKVDAKNALE NYAYNMRNTI KDEKIA...S KLDAADKKKI EDAIDQAIEW |
| Drosophila | QRIASRNALE SYVFNVKQAV EQAG.A...G KLDEADKNSV LEKCNETISW |
| saccharomyces | ERVQAKNQLE SYAFTLKNTI NEASFK...E KVGEDDAKRL ETASQETIDW |
| tuberculosisH37Rv | EEADVRNQAE TLVYQTEKFV KEQREAEGGS KVPEDTLNKV DAAVAEAKAA |
| leprae | EEADVRNQAE TLVYQTEKFV KEQRETENGS RVPEDTLNKV EAAVAEAKTA |
| Staph | EEVDLRNEAD SLVFQVEKTL .....TDLGE NIGEEDKKSA EEKKDALKTA |
| Ecoli | ELVQTRNQGD HLLHSTRKQV E.....EAGD KLPADDKTAI ESALTALETA |
| | 601 650 |
| Mouse | LDSNTLADKE EFVHKREELE RVCSPIISGL Y.QGAGA.PG ...AGGF... |
| Rat | LDSNTLAEKE EFVHKREELE RVCNPIISGL Y.QGAGA.PG ...AGGF... |
| bovine | LDANTLAEKD EFEHKRKELE QVCNPIISRL Y.QGAGG.PG ...AGGF... |
| human | LDANTLAEKD EFEHKRKELE QVCNPIISGL Y.QGAGG.PG ...PGGF... |
| Xenopus | LENNQLAEKE EYAFQQKDLE KVCQPIITKL Y.QG.GV.PG .GVPGGMPGS |
| Arabidopsis | LDGNQLAEAD EFEDKMKELE SLCNPIIARM Y.QGAGP.DM .GGAGGMDDD |
| Drosophila | LDSNTTAEKE EFDHRLEELT RHCSPIMTKM HQQGAGA... ..QAGGGPGA |
| saccharomyces | LDASQAASTD EYKDRQKELE GIANPIMTKF YGAGAGAGPG AGESGGFPGS |
| tuberculosisH37Rv | LGGS...DIS AIKSAMEKLG QESQALGQAI YEAAQAAS.. .........Q |
| leprae | LGGT...DIS AIKSAMEKLG QDSQALGQAI YEATQAAS.. .........K |
| Staph | LEGQ...DIE DIKSKKEELE KVIQELSAKV YE..QAAQ.. .........Q |
| Ecoli | LKGE...DKA AIEAKMQELA QVSQKL.MEI AQQQHAQQ.. .........Q |
| | 651 686 |
| Mouse | ..GAQAPKGA S.G.SGPTIE EVD*------ ------ |
| Rat | ..GAQAPKGG S.G.SGPTIE EVD------- ------ |
| bovine | ..GAQGPKGG S.G.SGPTIE EVD*------ ------ |
| human | ..GAQGPKGG S.G.SGPTIE EVD*------ ------ |
| Xenopus | SCGAQARQGG N...SGPTIE EVD------- ------ |
| Arabidopsis | T.....PAGG SGG.AGPKIE EVD*------ ------ |
| Drosophila | NCGQQA..GG FGGYSGPTVE EVD*------ ------ |
| saccharomyces | MPNSGATGGG ED..TQPTVE EVD*------ ------ |
| tuberculosisH37Rv | ATGAAHPGGE PGGAHPGSAD DVVDAEVVDD GREAK* |
| leprae | VGGEA...SA PGGSN..STD DVLTRRWSTT NGSPK* |
| Staph | Q..QQAQGAN AGQNNDSTVE DAEFKEVKDD DKK*-- |
| Ecoli | TAGA...DAS ANNAKDDDVV DAEFEEVKDK K----- |

DISTANCES between protein sequences in: HSP70-proteins.msf(*)

Correction method: Simple distance (no corrections)
Distances are: observed number of substitutions per 100 amino acids
Sy\acute{m}matrix version 1
Number of matrices: 1
Matrix 1, dimension: 12

Key for column and row indices:

| | |
|---|---|
| 1 | Mouse |
| 2 | Rat |
| 3 | bovine |
| 4 | human |
| 5 | Xenopus laevis |
| 6 | Arabidopsis thaliana |
| 7 | Drosophila |
| 8 | Saccharomyces cerevisiae |
| 9 | Mycobacterium tuberculosis H37Rv |
| 10 | Mycobacterium leprae |
| 11 | Staphylococcus aureus |
| 12 | E. coli DnaK |

Matrix 1: Part 1

| | 1 | 2 | 3 | 4 | 5 | 6 | 7 | 8 | 9 | 10 | 11 | 12 |
|---|---|---|---|---|---|---|---|---|---|---|---|---|
| 1 | 0.00 | 1.72 | 4.52 | 4.83 | 14.55 | 24.45 | 22.78 | 27.27 | 50.92 | 50.85 | 50.77 | 50.74 |
| 2 | | 0.00 | 3.59 | 3.74 | 13.93 | 24.33 | 22.98 | 26.69 | 51.01 | 51.10 | 50.34 | 50.82 |
| 3 | | | 0.00 | 1.40 | 14.24 | 23.35 | 24.05 | 25.71 | 51.09 | 51.02 | 50.94 | 50.08 |
| 4 | | | | 0.00 | 13.93 | 23.51 | 23.89 | 25.55 | 51.09 | 51.02 | 50.94 | 50.08 |
| 5 | | | | | 0.00 | 25.08 | 26.14 | 25.66 | 52.10 | 52.03 | 50.69 | 50.08 |
| 6 | | | | | | 0.00 | 30.50 | 29.91 | 52.19 | 52.03 | 53.86 | 51.39 |
| 7 | | | | | | | 0.00 | 29.78 | 51.01 | 50.59 | 51.54 | 51.22 |
| 8 | | | | | | | | 0.00 | 50.08 | 49.83 | 50.00 | 51.14 |
| 9 | | | | | | | | | 0.00 | 8.37 | 41.08 | 43.02 |
| 10 | | | | | | | | | | 0.00 | 41.42 | 43.40 |
| 11 | | | | | | | | | | | 0.00 | 41.43 |
| 12 | | | | | | | | | | | | 0.00 |

What is claimed:

1. A heat shock protein fragment that can increase the level of one or more cytokines and/or one or more CC chemokines and/or NO produced by a cell, above that caused by the corresponding full length heat shock protein.

2. A heat shock protein fragment according claim 1 that is a fragment of a human heat shock protein.

3. A heat shock protein according to claim 1 wherein the heat shock protein fragment is less than 80% of the size of the corresponding full length heat shock protein.

4. A heat shock protein fragment according any of claims 1 that is a fragment of a human HSP70.

5. A heat shock protein fragment according to any of claims 1 wherein the fragment has at least 40% homology to amino acid residues 359-625 or 359-610 of Mycobacterium tuberculosis HSP70.

6. A heat shock protein fragment according to any of claims 1 wherein the fragment has at least 60% homology to amino acid residues 359-459 of Mycobacterium tuberculosis HSP70.

7. A heat shock protein fragment according to any of claims 1 wherein the fragment has at least 80% homology to amino acid residues 396-426 of Mycobacterium tuberculosis HSP70.

8. A heat shock protein fragment consisting of amino acid residues 359-625, 359-610, 359-459, or 396-426 of Mycobacterium tuberculosis HSP70.

9. A heat shock protein fragment according claim 1 wherein the one or more cytokines are selected from the group consisting of interleukins and TNF-α.

10. A heat shock protein fragment according to claim 10 wherein the one or more chemokines are RANTES, MIP-α, or MIP-β.

11. A heat shock protein fragment according to claim 9 wherein the cytokines are IL-12 and/or TNF-α.

12. A heat shock protein fragment according to claim 1 that comprises a CD40 binding site.

13. A heat shock protein fragment according to claim 1 which additionally comprises one or more heterologous peptides.

14. A heat shock protein fragment according to claim 14 wherein the one or more heterologous peptides are immunogenic peptides.

15. An isolated nucleic acid molecule encoding the heat shock protein fragment according to claim 1.

16. A vector comprising the nucleic acid molecule of claim 15.

17. A host cell comprising the vector of claim 16.

18. A pharmaceutical composition comprising the heat shock protein fragment of claim 1 or the nucleic acid of claim in combination with a pharmaceutically acceptable excipient, carrier, adjuvant or vehicle.

19. The use of the heat shock protein fragment of claim 1 in therapy.

20. The use of the heat shock protein fragment of claim 1 in the manufacture of a medicament for the treatment or prophylaxis of a disease.

21. A method of treatment or prophylaxis of a disease, comprising administering to a patient in need, an effective dose of the heat shock protein fragment of claim 1.

22. The use of claim 20, wherein the disease is a microbial infection, a viral infection, a disease of the immune system or a cancer.

23. A method of increasing production of one or more cytokines and/or one or more CC chemokines and/or NO above the level of production brought about by the corresponding full length heat shock protein comprising contacting a cell with the heat shock protein fragment of claim 1.

24. The use of the heat shock protein fragment of claim 1 to increase the production of one or more cytokines and/or one or more CC chemokines and/or NO above the level brought about by the corresponding full length heat shock protein.

25. The use of the heat shock protein fragment of claim 1 to polarize an immune response towards a Th1 response.

26. A heat shock protein fragment according to claim 1 in combination with a vaccine.

27. The use according to any of claim 25 wherein the heat shock protein is used in combination with a vaccine.

28. A polypeptide comprising amino acid residues 359-625 of the C-terminal region of the heat shock protein HSP70.

29. A polypeptide comprising amino acid residues 359-610 of the C-terminal region of the heat shock protein HSP70.

30. An adjuvant comprising a polypeptide according to claim 28.

31. An adjuvant according to claim 30, connected covalently or non-covalently to an antigen.

32. A vaccine comprising an adjuvant according to claim 31.

33. A vaccine against HIV comprising an adjuvant according to claim 31.

34. A DNA molecule coding for a polypeptide according to claim 28.

35. A DNA molecule according to claim 34, having the sequence given in FIG. 4.

36. A heat shock protein fragment according to claim 8 wherein the one or more cytokines are selected from the group consisting of interleukins and TNF-α.

37. A heat shock protein fragment according to claim 8 that comprises a CD40 binding site.

38. A heat shock protein fragment according to claim 8 which additionally comprises one or more heterologous peptides.

39. An isolated nucleic acid molecule encoding the heat shock protein fragment according to claim 8.

40. A pharmaceutical composition comprising the heat shock protein fragment of claim 8 or the nucleic acid of claim 15 in combination with a pharmaceutically acceptable excipient, carrier, adjuvant or vehicle.

41. The use of the heat shock protein fragment of claim 8 in therapy.

42. The use of the heat shock protein fragment of claim 8 in the manufacture of a medicament for the treatment or prophylaxis of a disease.

43. A method of treatment or prophylaxis of a disease, comprising administering to a patient in need, an effective dose of the heat shock protein fragment of claim 8.

44. The use of claim 21, wherein the disease is a microbial infection, a viral infection, a disease of the immune system or a cancer.

45. A method of increasing production of one or more cytokines and/or one or more CC chemokines and/or NO above the level of production brought about by the corresponding full length heat shock protein comprising contacting a cell with the heat shock protein fragment of claim 8.

46. The use of the heat shock protein fragment of claim 8 to increase the production of one or more cytokines and/or one or more CC chemokines and/or NO above the level brought about by the corresponding full length heat shock protein.

47. The use of the heat shock protein fragment of claim 8 to polarize an immune response towards a Th1 response.

48. A heat shock protein fragment according to claim 8 in combination with a vaccine.

49. The use according to claim 26 wherein the heat shock protein is used in combination with a vaccine.

50. An adjuvant comprising a polypeptide according to claim 29.

51. A DNA molecule coding for a polypeptide according to claim 29.
